(19) United States
(12) Patent Application Publication
(10) Pub. No.: US 2016/0246259 A1
(43) Pub. Date: Aug. 25, 2016

(54) METHOD, APPARARUS AND WEARABLE APPARATUS FOR AUTOMATICALLY REMINDING USER TO SLEEP

(71) Applicant: HUIZHOU TCL MOBILE COMMUNICATION CO., LTD., HuiZhou City, Guangdong (CN)

(72) Inventor: Jie ZHANG, HuiZhou City (CN)

(21) Appl. No.: 14/438,881

(22) PCT Filed: May 28, 2014
(86) PCT No.: PCT/CN2014/078610

§ 371 Date: Apr. 28, 2015

(30) Foreign Application Priority Data
Feb. 28, 2014 (CN) 201410070723.9

(51) Int. Cl. G04B 23/12; A61B 5/00; A61B 5/11; G08B 21/24

(52) U.S. Cl. CPC G04B 23/12; G08B 21/24; A61B 5/486; A61B 5/1118; A61B 5/4809; A61B 5/4815

(57) ABSTRACT

In the present invention, information about daily activities is acquired, cumulative intensity of present-day activities is calculated, the required sleeping duration is calculated based on the cumulative intensity of the present-day activities and the functional relationship between the cumulative intensity of the daily activities and the sleeping duration, a set waking alarm clock time is acquired, the user's starting bedtime is calculated by using the waking alarm clock time and the required sleeping duration of the user, and the user is reminded to sleep when the starting bedtime is reached. The user is reminded in advance by analyzing the daily activities and records to determine the optimal sleeping duration.

### FIELD OF THE INVENTION

The present invention relates to the field of consumer electronics, and in particular to a method, a system, and a wearable apparatus for automatically reminding a user to sleep by analyzing the user's daily activities.

### BACKGROUND OF THE INVENTION

With the development of electronic technology and the continuous improvement of people's living standards, the application of the wearable consumer electronic products, such as smart watches and smart bracelets, has become increasingly popular. The basic functions of these electronic products generally include monitoring sleeping, walking, sitting, and other user activity via built-in sensors, as well as achieving alarms via motors and speakers.

Alerting the user when to get up with an alarm has become a necessary function. However, it is more important to inform the user when to sleep if the user wants to get up on time and be energetic the next day. Nevertheless, the wearable apparatus in the prior art does not have a function to automatically remind the user to sleep by analyzing the user's daily activities.

Therefore, the prior art can still be improved and developed.

### SUMMARY OF THE INVENTION

The present invention resolves the technical problem by aiming to solve the deficiency above in the prior art by providing a method, a system, and a wearable apparatus for automatically reminding a user to sleep by analyzing the user's daily activities. An optimal sleeping duration is determined in advance for reminding the user through a built-in sensor and alarm function of a wearable consumer electronic product by analyzing the user's daily activities and past historical records.

The technical solutions used in present invention for resolving the technical problems are as follows.

A method for automatically reminding a user to sleep, comprising steps of:

acquiring and storing information about the user's daily activities, and calculating the cumulative intensity of the user's present-day activities based on the acquired information about the user's daily activities, wherein the information about the daily activities include walking, running, biking, and taking a vehicle;
acquiring historical data of the user's daily activities, and establishing a functional relationship between the cumulative intensity of the daily activities and a sleeping duration based on the historical data of the user's daily activities;
calculating the required sleeping duration of the user based on the cumulative intensity of the present-day activities and the functional relationship between the cumulative intensity of the daily activities and the sleeping duration;
acquiring a waking alarm clock time which is set by the user, and calculating the user's starting bedtime by using the waking alarm clock time and the required sleeping duration of the user; and
reminding the user to sleep when the starting bedtime is reached.

The technical solutions used in present invention for resolving the technical problems are as follows.

A method for automatically reminding a user to sleep, comprising steps of:

acquiring and storing information about the user's daily activities, and calculating cumulative intensity of the user's present-day activities based on the acquired information about the user's daily activities;
calculating the required sleeping duration of the user based on the cumulative intensity of the present-day activities and the functional relationship between the cumulative intensity of the daily activities and the sleeping duration;
acquiring a waking alarm clock time which is set by the user, and calculating the user's starting bedtime by using the waking alarm clock time and the required sleeping duration of the user; and
reminding the user to sleep when the starting bedtime is reached.

The technical solutions used in present invention for resolving the technical problems are as follows.

A system for automatically reminding a user to sleep, comprising:

a present-day activity cumulative intensity calculating module for acquiring and storing information about the user's daily activities, and for calculating the cumulative intensity of the user's present-day activities based on the information about the user's daily activities;
a required sleeping duration calculating module for calculating the required sleeping duration of the user based on the cumulative intensity of the present-day activities and the functional relationship between the cumulative intensity of the daily activities and the sleeping duration;
a starting bedtime calculating module for acquiring a waking alarm clock time which is set by the user, and calculating the user's starting bedtime by using the waking alarm clock time and the required sleeping duration of the user; and
an reminding control module for reminding the user to sleep when the starting bedtime is reached.

The technical solutions used in present invention for resolving the technical problems are as follows.

A wearable apparatus including a system for automatically reminding a user to sleep, the system comprising:

a present-day activity cumulative intensity calculating module for acquiring and storing information about the user's daily activities, and for calculating the cumulative intensity of the user's present-day activities based on the information about the user's daily activities;
a required sleeping duration calculating module for calculating the required sleeping duration of the user based on the cumulative intensity of the present-day activities and the functional relationship between the cumulative intensity of the daily activities and the sleeping duration;
a starting bedtime calculating module for acquiring a waking alarm clock time which is set by the user, and calculating the user's starting bedtime by using the waking alarm clock time and the required sleeping duration of the user; and
an reminding control module for reminding the user to sleep when the starting bedtime is reached.

The present invention provides a method, a system, and a wearable apparatus for automatically reminding a user to sleep by analyzing the user's daily activities, so as to add a new function, which automatically reminds the user when to sleep by analyzing the user's daily activities, to the wearable apparatus. An optimal sleeping duration is determined in advance and combined with an alarm function for automatically reminding the user through a built-in sensor and alarm function of a wearable consumer electronic product by analyzing the user's daily activities and past historical record.

### BRIEF DESCRIPTION OF THE DRAWINGS

FIG. 1 is a flow chart in accordance with a method for automatically reminding a user to sleep of a preferably embodiment of the present invention;

FIG. 2 is a waveform diagram during walking in accordance with a method for automatically reminding a user to sleep of a preferably embodiment of the present invention;

FIG. 3 is a schematic diagram of a curve structure of a positive correlation between sleeping durations and cumulative intensity of the daily activities in accordance with a method for automatically reminding a user to sleep of a preferably embodiment of the present invention;

FIG. 4 is a block diagram of functions and principles based on a remote processing system of a mobile phone in accordance with a preferably embodiment of the present invention.

### DETAILED DESCRIPTION OF THE PREFERRED EMBODIMENTS

Please refer to the drawings, wherein the components with the same reference numbers represent the same components. The principle of the present invention is implemented in a suitable calculating environment for illustrative purposes. The following description is based on the illustrated specific embodiment of the present invention, and should not be construed to limit other specific embodiments of the present invention, which are not described in detail herein.

In the following description, the specific embodiments of the present invention are described with reference to reference numbers or steps executed by one or more calculators, unless stated otherwise. Therefore, it will be mentioned several times that these steps and operations are executed by the computers. The execution of the computer herein includes an operation of a processing unit representing the computer for electronic signals of data in a structured pattern. Such an operation converts the data or maintains them on the location of the memory system of the computer, it can be reconfigured, or the operation of the computer can be altered in a manner known by a person skilled in the art. The maintained data structure of the data is the physical location of the memory, which has a specific characteristic defined by the data format. However, the principles of the present invention described above do not represent a limitation. A person skilled in the art can understand that various steps and operations described below can be implemented in hardware.

The principle of the present invention is operated by using a number of other calculations, communication environments, or configurations of a general or special purpose. Well-known examples of the calculating systems, environments and configurations suitable for the present invention include (but are not limited to) mobile phones, personal computers, servers, multiprocessor systems, microcomputer-based systems, main frame computers, and distributed calculating environments, which includes any system or apparatus described above.

The term “module” used herein can be regarded as a software object executed on the calculating system. The different components, modules, engines, and services described herein can be regarded as an object implemented on the calculating system. The apparatus and method described herein are preferably implemented in a software manner, and certainly can also be implemented on hardware, which are both within the scope of the present invention.

Wearable consumer electronics, which are micro-electronic devices which can be worn while the user does activities, include smart watches, health bracelets, and smart bracelets, which are well-known by the public, and usually composed of low-power microcontrollers, sensors, motors, batteries, Bluetooth antennas and other components. Smart watches generally have a low-power display. The main features of wearable consumer electronic products include a long standby time, so as to usually work for one week or more. The user can continuously wear them during sleep and rest.

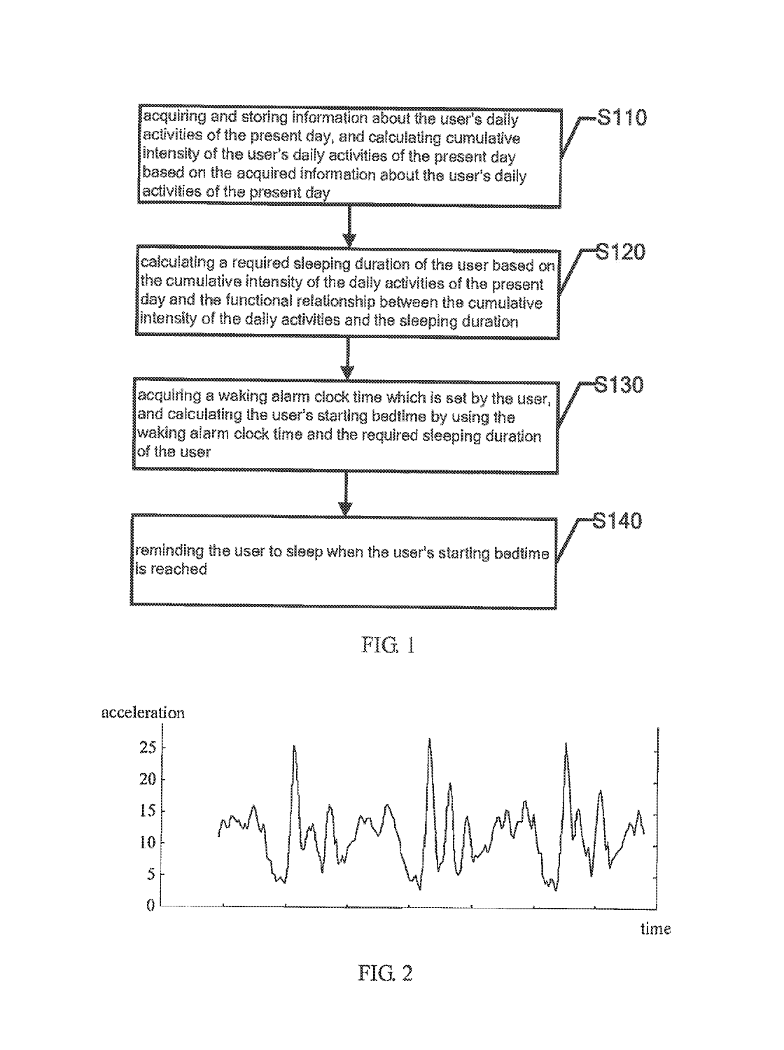

Please refer to FIG. 1, which is a flow chart in accordance with a method for automatically reminding a user to sleep of a preferably embodiment of the present invention. As shown in FIG. 1, the method for automatically reminding a user to sleep comprises the steps of:

In Step S110, information about the user's daily activities is acquired and stored, cumulative intensity of the user's present-day activities is calculated based on the acquired information about the user's daily activities;

The step S110 specifically includes steps of:

(A1) acquiring the information about the user's present-day daily activities by acquiring accelerations of various actions of the user's present-day activities via a sensor;
(A2) determining intensity of each activity based on the acquired information about the daily activities; and
(A3) calculating the cumulative intensity of the user's present-day activities based on the determined intensity of each activity, wherein the information about the daily activities includes walking, running, biking, and taking a vehicle.

In the embodiment of the present invention, firstly, the user's information about the daily activities is acquired and stored, for example, by using a variety of sensors which can accurately determine the user's current behavior, such as sleeping, walking, running, biking, taking a vehicle, etc, and also can determine the intensity of the walking, running, biking, and other activities.

The sensors in the embodiment are devices receiving signals or stimuli, or devices converting to-be-tested physical quantity or chemical quantity into another corresponding output, and include, but are not limited to, acceleration sensors, pressure sensors, and temperature sensors.

Theoretically, the more the sensors that are used, the more comprehensive the data that can be detected, certainly, the more accurately the user's behavior can be detected. There are many sophisticated algorithms used for the detection and the analysis of the user's behavior. Currently, very reliable information can be acquired only based upon the data of the acceleration sensors.

When the user is doing activity, even a slight movement can be detected by the acceleration sensors. The data acquired by the sensors are the acceleration of the movement. When the user is walking, the frequency of the swinging arms is generally about 1.5 times per second. If the horizontal axis is time, and the vertical axis is the acceleration, the waveform as shown in FIG. 2 can be acquired.

Therefore, the user's current behavior can be accurately determined by recording the returning data of the acceleration sensors in a sufficient time (such as 30 to 60 seconds), and comparing it with the established characteristic data of a typical movement. For example, pedometers well-known by sport enthusiasts are based on this principle.

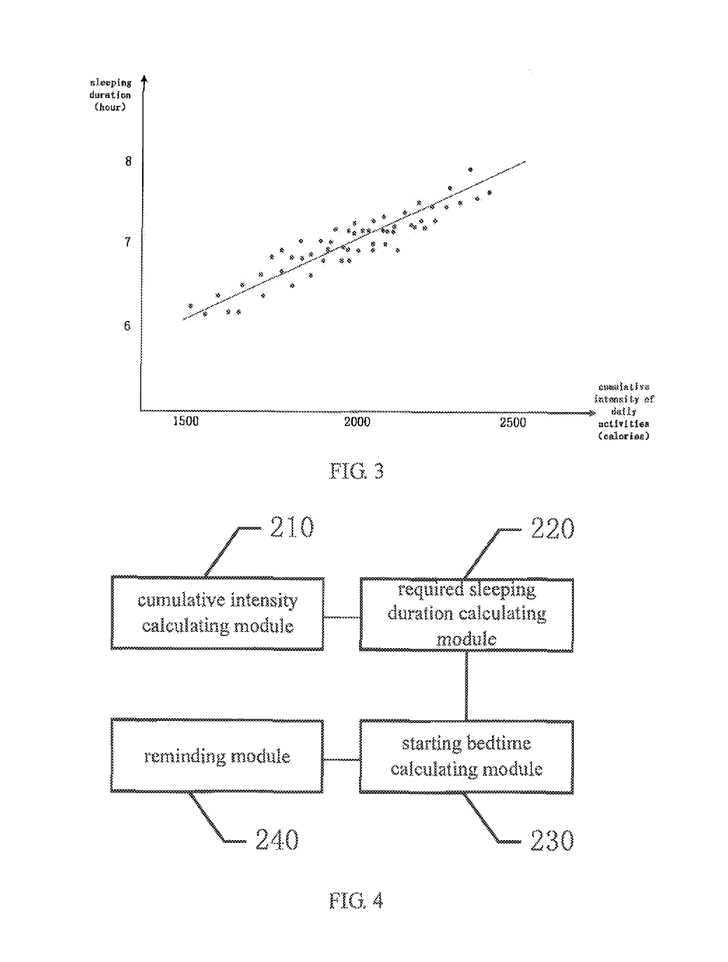

Although each person's health status, age, weight, and other factors are different, and the sleeping duration needed daily is also affected by diet and mental status, the most important determinant of the needed sleeping duration is still the activity intensity of the present day, as shown in FIG. 3. FIG. 3 is a schematic diagram of a curve structure of a positive correlation between sleeping durations and cumulative intensity of the daily activities in accordance with a method for automatically reminding a user to sleep of a preferably embodiment of the present invention. In FIG. 3, although the collected daily data shown are scattered data, a gradual positive correlation curve can be drawn with the increase of the sample size.

The calorie calculation of the intensity of the activities is explained herein. Although the calorie consumption of each person doing the same activity, such as walking, is different due to weight, age, sex, and other factors, the relative values between different activities are emphasized herein, and the absolute values have no effect on the calculation results of the present invention. Therefore, for the same activity of different users, the same typical calorie consumption values can be used in the calculation.

In the calculation method, the user's present activity status at every fixed time interval (typically 60 seconds) is determined by using a timer, typical calorie consumptions are acquired based on different activities, and the cumulative intensity of the user's present-day activities are accordingly cumulatively calculated.

Typical caloric consumption values (calories/hour) as shown in the following table:

| | Low intensity | Moderate intensity | High intensity |
|---|---|---|---|
| Walking | 255 | 555 | |
| Running | 655 | 700 | |
| Biking | 245 | 435 | 655 |
| Sitting, Reading, | 65 | 76 | 90 |
| Meeting, etc. | | | |

Afterwards, the procedure proceeds to step S120.

In step S120, the required sleeping duration of the user is calculated based on the cumulative intensity of the present-day activities and the functional relationship between the cumulative intensity of the daily activities and the sleeping duration.

In the embodiment of the present invention, before the establishment of the functional relationship between the cumulative intensity of the former daily activities and a sleeping duration, the historical data of the user's daily activities is required.

The historical data of the user's daily activities can be acquired by acquiring the sleeping duration and the cumulative intensity of the daily activities by detecting a permanent memory, storing the same into a local storage space or a remote server, and acquiring the historical data of the daily activities.

The more activities the user does, the more tired the user feels, the longer sleeping duration the user needs. By monitoring the sleeping duration every day in the past and the activity intensity of the user in the past the specific functional relationship between the sleeping duration and the activity intensity of the user can be accurately established (correspondence table). Therefore, eventually, the following function can be obtained from the historical data:

Sleeping duration=f(cumulative intensity of the daily activities), where f is a natural number. In case the historical data are not enough to obtain this function, it can temporarily be replaced with a default function.

By monitoring the sleeping duration every day in the past and the historical data of the daily activities of the user, the specific functional relationship between the sleeping duration and the activity intensity of the user is established, the sleeping duration=f(the cumulative intensity of the daily activities), wherein f is a natural number.

The required sleeping duration of the user is calculated based on the cumulative intensity of the present-day activities and the functional relationship between the cumulative intensity of the daily activities and the sleeping duration. For instance, if the user runs for a long time today, the intensity is 700. When f is set to be 0.015, the required sleeping duration can be calculated as follows: sleeping duration=f(cumulative intensity of the daily activities)=0.015*700=10.5 hours, that is, the required sleeping duration of the user is 10 and half hours.

In step S130, a waking alarm clock time which is set by the user is acquired, and the user's starting bedtime is calculated by using the waking alarm clock time and the required sleeping duration of the user.

In the embodiment of this step, the waking alarm clock time set by the user is acquired first, such as 7:00 AM. Afterwards, the user's starting bedtime is calculated by using the waking alarm clock time and the required sleeping duration of the user: (7.00+24)−10.5=20.5, that is, the user's starting bedtime calculated should be 20:30 PM, that is, 20:30 PM is the user's starting bedtime.

Furthermore, after lying on the bed, the user may not fall asleep right away, so a transition period is required. After the alarm clock time set by the user is acquired, the alarm clock application requires the following information:
(1) How much time is left from the present time to the waking alarm clock time set by the user? (The information is defined as the available sleeping duration.)
(2) The required sleeping duration of the user can be calculated based on the cumulative intensity of the present-day activities of the user so far and the functional relationship. (The information is defined as the required sleeping duration)

A typical transition period from getting ready to sleep to going to bed is required to be defined, such as half an hour.

By monitoring, it is found that:

the available sleeping duration=the required duration+the transition period. The available sleeping duration=the required sleeping duration (10.5 hour)+the transition period (0.5 hour)=11 hour.

In step S140, the user is reminded to sleep when the starting bedtime is reached.

As mentioned above, if 20:30 PM is the user's starting bedtime, when the starting bedtime time of 20:30 is reached, the user is automatically reminded to sleep by sounds or vibrations with text display.

Based upon the aforementioned embodiment, the present invention also provides a system automatically reminding the user to sleep, as shown in FIG. 4, comprising: a present-day activity cumulative intensity calculating module 410 used for acquiring and storing information about the user's daily activities, and for calculating the cumulative intensity of the user's present-day activities based on the information about the user's daily activities, as specifically described above;

a required sleeping duration calculating module 420 used for calculating the required sleeping duration of the user based on the cumulative intensity of the present-day activities and the functional relationship between the cumulative intensity of the daily activities and the sleeping duration, as specifically described above;
a starting bedtime calculating module 430 used for acquiring a waking alarm clock time which is set by the user, and calculating the user's starting bedtime by using the waking alarm clock time and the required sleeping duration of the user, as specifically described above; and
an reminding control module 440 used for reminding the user to sleep when the starting bedtime is reached, as specifically described above.

The present-day activity cumulative intensity calculating module 410 comprises:

a sensor control processing unit for acquiring the information about the user's present-day daily activities by acquiring accelerations of various actions of the user's present-day activities through a sensor, as specifically described above;
a determination processing unit for determining intensity of each activity by acquiring the information about the daily activities, as specifically described above; and
a present-day activity cumulative intensity calculating unit for controlling a timer, for determining the user's present activity status at every fixed time interval, for acquiring typical calorie consumptions based on different activities, and for accordingly cumulatively calculating the cumulative intensity of the user's present-day activities, as specifically described above.

The required sleeping duration calculating module comprises:

a daily activity historical data calculating unit for acquiring the sleeping duration and the cumulative intensity of the daily activities by detecting by a permanent memory, storing the same into the a local storage space or a remote server, and acquiring the historical data of the daily activities, as specifically described above;
a sleeping duration function determining unit for establishing the specific functional relationship between the sleeping duration and the activity intensity of the user, the sleeping duration=f(the cumulative intensity of the daily activities), by monitoring the sleeping duration every day in the past and the historical data of the daily activities of the user, wherein f is a natural number, as specifically described above; and
a required sleeping duration calculating unit for calculating the required sleeping duration of the user based on the cumulative intensity of the present-day activities and the functional relationship between the cumulative intensity of the daily activities and the sleeping duration, as specifically described above.

Based on the aforementioned embodiments, the present invention also provides a wearable device, comprising the system automatically reminding the user to sleep described in the aforementioned embodiment.

In summary, the present invention provides a method, a system, and a wearable apparatus for automatically reminding a user to sleep by analyzing the user's daily activities, so as to add a new function, which automatically reminds the user when to sleep by analyzing the user's daily activities, to the wearable apparatus. An optimal sleeping duration is determined and combined with an alarm function for automatically reminding the user in advance through a built-in sensor and alarm function of a wearable consumer electronic product by analyzing the user's daily activities and past historical records.

It should be understood that the application of the present invention is not limited to the above embodiments, which can be modified or altered by those persons of ordinary skill in the art according to the above descriptions. All such modifications and alterations fall within the scopes of the appended claims of the invention.

1. A method for automatically reminding a user to sleep, comprising steps of:
acquiring and storing information about the user's daily activities of the present day, and calculating cumulative intensity of the user's daily activities of the present day based on the acquired information about the user's daily activities of the present day;
acquiring historical data of the user's daily activities, and establishing a functional relationship between the cumulative intensity of the daily activities and a sleeping duration based on the historical data of the user's daily activities;
calculating a required sleeping duration of the user based on the cumulative intensity of the daily activities of the present day and the functional relationship between the cumulative intensity of the daily activities and the sleeping duration;
acquiring a waking alarm clock time which is set by the user, and calculating the user's starting bedtime by using the waking alarm clock time and the required sleeping duration of the user; and
reminding the user to sleep when the user's starting bedtime is reached.

2. The method for automatically reminding a user to sleep according to claim 1, wherein the step of acquiring and storing information about the user's daily activities of the present day, and calculating cumulative intensity of the user's daily activities of the present day based on the acquired information about the user's daily activities of the present day comprise steps of:
acquiring the information about the user's present-day daily activities by acquiring accelerations of various actions of the user's daily activities of the present day via a sensor;
determining intensity of each daily activity based on the acquired information about the user's daily activities of the present day, and
calculating the cumulative intensity of the user's daily activities of the present day based on the determined intensity of each daily activity.

3. The method for automatically reminding a user to sleep according to claim 1, wherein the step of acquiring and storing information about the user's daily activities of the present day, and calculating cumulative intensity of the user's daily activities of the present day based on the acquired information about the user's daily activities of the present day further comprise steps of:
determining the user's present activity status at every fixed time interval by using a timer, acquiring typical calorie consumptions based upon the different daily activities, and cumulatively calculating the cumulative intensity of the user's daily activities of the present day based on the typical calorie consumptions.

4. The method for automatically reminding a user to sleep according to claim 2, wherein the step of acquiring and storing information about the user's daily activities of the present day, and calculating cumulative intensity of the user's daily activities of the present day based on the acquired information about the user's daily activities of the present day further comprise steps of:
determining the user's present activity status at every fixed time interval by using a timer, acquiring typical calorie consumptions based upon the different daily activities, and cumulatively calculating the cumulative intensity of the user's daily activities of the present day based on the typical calorie consumptions.

5. The method for automatically reminding a user to sleep according to claim 1, wherein the step of acquiring historical data of the user's daily activities comprises steps of:
acquiring the sleeping duration and the cumulative intensity of the daily activities by detecting a permanent memory, storing the same into the a local storage space or a remote server, and acquiring the historical data of the daily activities.

6. The method for automatically reminding a user to sleep according to claim 1, wherein the step of acquiring historical data of the user's daily activities, and establishing a functional relationship between the cumulative intensity of the daily activities and a sleeping duration comprise a step of:
establishing the functional relationship between the sleeping duration and the activity intensity of the specific user, as: the sleeping duration=f(the cumulative intensity of the daily activities), by monitoring the sleeping duration every day in the past and acquiring the historical data of the daily activities of the user, wherein f is a natural number.

7. The method for automatically reminding a user to sleep according to claim 4, wherein the step of acquiring historical data of the user's daily activities comprises steps of:
acquiring the sleeping duration and the cumulative intensity of the daily activities by detecting a permanent memory, storing the same into the a local storage space or a remote server, and acquiring the historical data of the daily activities.

8. The method for automatically reminding a user to sleep according to claim 4, wherein the step of acquiring historical data of the user's daily activities, and establishing a functional relationship between the cumulative intensity of the daily activities and a sleeping duration comprise a step of:
establishing the functional relationship between the sleeping duration and the activity intensity of the specific user, the sleeping duration=f(the cumulative intensity of the daily activities), by monitoring the sleeping duration every day in the past and acquiring the historical data of the daily activities of the user, wherein f is a natural number.

9. (canceled)

10. (canceled)

11. (canceled)

12. (canceled)

13. (canceled)

14. (canceled)

15. A system for automatically reminding a user to sleep, comprising:
a cumulative intensity calculating module for acquiring and storing information about the user's daily activities of the present day, and for calculating the cumulative intensity of the user's daily activities of the present day based on the information about the user's daily activities of the present day;
a required sleeping duration calculating module for calculating a required sleeping duration of the user based on the cumulative intensity of the user's daily activities of the present day and the functional relationship between the cumulative intensity of the daily activities and the sleeping duration;
a starting bedtime calculating module for acquiring a waking alarm clock time which is set by the user, and for calculating the user's starting bedtime by using the waking alarm clock time and the required sleeping duration of the user; and
a reminding module for reminding the user to sleep when the user's starting bedtime is reached.

16. The system for automatically reminding a user according to claim 15, wherein the cumulative intensity calculating module comprises:
a sensor control unit for acquiring the information about the user's daily activities of the present day by acquiring accelerations of various actions of the user's present-day activities through a sensor,
a determination unit for determining intensity of each daily activity by acquiring the information about the daily activities; and
a cumulative intensity calculating unit for controlling a timer, for determining the user's present activity status at every fixed time interval, for acquiring typical calorie consumptions based on the different daily activities, and for cumulatively calculating the cumulative intensity of the user's daily activities of the present day based on the typical calorie consumptions.

17. The system for automatically reminding a user according to claim 15, wherein the required sleeping duration calculating module comprises:
a historical data calculating unit for acquiring the sleeping duration and the cumulative intensity of the daily activities by detecting a permanent memory, storing the same into the a local storage space or a remote server, and acquiring the historical data of the daily activities;
a function determining unit for establishing the functional relationship between the sleeping duration and the activity intensity of the specific user, the sleeping duration=f(the cumulative intensity of the daily activities), by monitoring the sleeping duration every day in the past and acquiring the historical data of the daily activities of the user, wherein f is a natural number; and
a required sleeping duration calculating unit for calculating the required sleeping duration of the user based on the cumulative intensity of the user's daily activities of the present day and the functional relationship between the cumulative intensity of the daily activities and the sleeping duration.

18. A wearable apparatus including a system for automatically reminding a user to sleep, the system for automatically reminding a user to sleep comprising:
a cumulative intensity calculating module for acquiring and storing information about the user's daily activities of the present day, and for calculating the cumulative intensity of the user's daily activities of the present day based on the information about the user's daily activities of the present day;
a required sleeping duration calculating module for calculating a required sleeping duration of the user based on the cumulative intensity of the user's daily activities of the present day and the functional relationship between the cumulative intensity of the daily activities and the sleeping duration;
a starting bedtime calculating module for acquiring a waking alarm clock time which is set by the user, and for calculating the user's starting bedtime by using the waking alarm clock time and the required sleeping duration of the user; and
a reminding module for reminding the user to sleep when the user's starting bedtime is reached.

19. The wearable apparatus according to claim 18, wherein the cumulative intensity calculating module comprises:
a sensor control unit for acquiring the information about the user's daily activities of the present day by acquiring accelerations of various actions of the user's present-day activities through a sensor;
a determination unit for determining intensity of each daily activity by acquiring the information about the daily activities; and
a cumulative intensity calculating unit for controlling a timer, for determining the user's present activity status at every fixed time interval, for acquiring typical calorie consumptions based on the different daily activities, and for cumulatively calculating the cumulative intensity of the user's daily activities of the present day based on the typical calorie consumptions.

20. The wearable apparatus according to claim 18, wherein the required sleeping duration calculating module comprises:
a historical data calculating unit for acquiring the sleeping duration and the cumulative intensity of the daily activities by detecting a permanent memory, storing the same into the a local storage space or a remote server, and acquiring the historical data of the daily activities;
a function determining unit for establishing the functional relationship between the sleeping duration and the activity intensity of the specific user, the sleeping duration=f(the cumulative intensity of the daily activities), by monitoring the sleeping duration every day in the past and acquiring the historical data of the daily activities of the user, wherein f is a natural number; and
a required sleeping duration calculating unit for calculating the required sleeping duration of the user based on the cumulative intensity of the user's daily activities of the present day and the functional relationship between the cumulative intensity of the daily activities and the sleeping duration.
